(19) United States
(12) Patent Application Publication
(10) Pub. No.: US 2025/0135125 A1
(43) Pub. Date: May 1, 2025

(54) INJECTION SYSTEM AND METHODS FOR FLEXIBLE MEDICAL DEVICES

(71) Applicant: NEURONOFF, INC., Cleveland, OH (US)

(72) Inventors: STEPHAN NIEUWOUDT, Cleveland, OH (US); ALAA ABD-ELSAYED, Cleveland, OH (US); AMOL SOIN, Cleveland, OH (US); EMILY SZABO, Cleveland, OH (US); AMY HOWE, Cleveland, OH (US); SHAHER AHMAD, Cleveland, OH (US); JENNIFER PECK, Cleveland, OH (US); KYLE GOLOBISH, Cleveland, OH (US); MANFRED FRANKE, CLEVELAND, OH (US)

(73) Assignee: NEURONOFF, INC., Cleveland, OH (US)

(21) Appl. No.: 18/835,430

(22) PCT Filed: Feb. 3, 2023
(86) PCT No.: PCT/US23/12348

§ 371 Date: Aug. 2, 2024

Related U.S. Application Data
(63) Continuation-in-part of application No. PCT/US2022/020652, filed on Mar. 16, 2022.
Application No. PCT/US2022/020652 is a continuation-in-part of application No. PCT/US2021/033265, filed on May 19, 2021.
Application No. PCT/US2021/033265 is a continuation-in-part of application No. PCT/US2021/033007, filed on May 18, 2021.
(60) Provisional application No. 63/400,725, filed on Aug. 24, 2022.
(60) Provisional application No. 63/400,709, filed on Aug. 24, 2022.
(60) Provisional application No. 63/319,379, filed on Mar. 13, 2022.
(60) Provisional application No. 63/306,896, filed on Feb. 4, 2022.
(60) Provisional application No. 63/306,397, filed on Feb. 3, 2022.

(51) Int. Cl. A61M 5/32; A61M 5/315

(52) U.S. Cl. CPC A61M 5/3287; A61M 5/31511

(57) ABSTRACT

A system and associated methods for injection of a substantially linear and flexible medical device within a body. The system uses markings on a needle and on a plunger to guide the clinician in withdrawing a needle and advancing a plunger to create 3D anchors out of a substantially linear device.

### PRIORITY STATEMENT

This application claims priority to U.S. provisional patent application No. 63/306,397 filed Feb. 3, 2022, U.S. provisional patent application No. 63/306,896 filed Feb. 4, 2022, U.S. provisional patent application No. 63/319,379 filed Mar. 13, 2022, international application #PCT/US2022/020652 filed Mar. 16, 2022, U.S. provisional patent application No. 63/400,709 filed Aug. 24, 2022, and U.S. provisional patent application No. 63/400,725 filed Aug. 24, 2022.

### INCORPORATION

The present application incorporates the following international applications: #PCT/US2021/033007 filed May 18, 2021 (“the '007 application”), #PCT/US2021/033265 filed May 19, 2021 (“the '265 application”), and #PCT/US2022/020,652 filed Mar. 16, 2022 (“the '652 application”) and adopts reference numbers from them.

### INTRODUCTION

Described herein is an injection system with associated methods for using the system to inject a flexible medical device 1 in a precise and controlled way near a tissue target including without limitation, a nerve, an organ, a muscle or a tumor. The medical device can be fully injected or partially injected leaving an external tail or an external loop whose end is also injected. The system and methods are configured for injecting any flexible medical device which is at least partially linear. The medical device may be intended for many purposes such as for, without limitation, neurostimulation, ablation, embolization, stents, and drug-eluting polymers.

### BRIEF DESCRIPTION OF THE FIGURES

FIG. 1(A)-(D) is a schematic show a flexible medical device with three regions producing two or three anchors and the relative length post-placement.

FIG. 2(A) is an image of a fully extended flexible medical device and FIGS. 2(B1), 2(B2) and 2(B3) are schematics showing relative lengths depending on the volume of the central anchor.

FIG. 3 are images of elements of the placement system in one embodiment.

FIG. 4 are images of needle tip configurations with or without a trocar.

FIG. 5 are schematics of a three region medical device and corresponding needle depth marks and plunger advance marks.

FIG. 6 are schematics showing withdrawal of the needle and advance of the plunger in various steps.

FIG. 7 contains multiple images of steps in the placement method on a benchtop.

FIG. 8(A)-(D) are schematics of a medical device with multiple distal regions pre-placement and post-placement.

FIG. 9(A)-(D) are schematics of a medical device with multiple proximal regions pre-placement and post-placement.

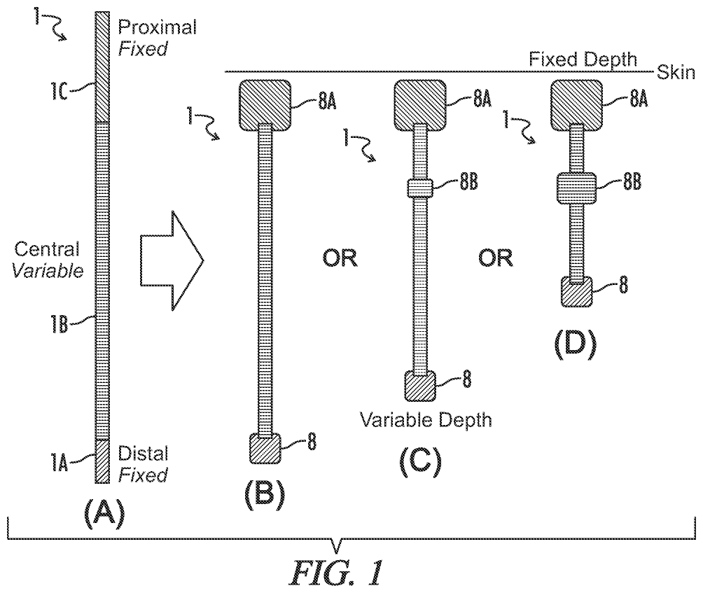

FIG. 1(A) is a schematic showing a medical device with multiple distal regions/marks and FIG. 10(B)-(C) are the associated markings on the needle and plunger.

FIG. 11(A) is a schematic showing a medical device with multiple proximal regions/marks and FIG. 11(B)-(C) are the associated markings on the needle and plunger.

FIG. 12 is a schematic showing a medical device post-placement in the body with two distal anchors and two proximal anchors (connector pads) and one central anchor.

FIG. 13 is a schematic of the plunger with securing clips for tactile sensing.

FIG. 14 is a schematic of the needle with projections for tactile sensing.

### ASPECTS OF THE INVENTION

In one embodiment, the system and methods are used to implant a flexible medical device which is a helical wire rope structure, also known as “Injectrode®” (as more fully described in the '007, '265 and '652 applications) into the body of an organism and allows for the controlled placement of regions of the device near at least one target tissue and elsewhere such as subcutaneous areas. The flexible medical device may be fully or partially injected. The system comprises a needle and a plunger with mathematically predetermined markings. The methods comprise the steps in which the needle and plunger are advanced and withdrawn concentrically during the placement. In one embodiment, this system and method places a fixed length flexible Injectrode® with three distinct regions: distal region 1A (fixed), central region 1B (variable), proximal region 1C (fixed). Here, the distal and proximal regions are fixed, whereas the central region is variable as regards the lengths of flexible medical device in each of these regions. Placing a variable length of device within the central region enables the placement of fixed length regions distally and proximally, at variable relative distance from one another, with the central region essentially retaining the slack of the Injectrode®, so as to aid in reducing tension on the device. This approach, as enabled by the placement system, takes a flexible medical device of a fixed length and adapts it to a variable target to target length.

In one embodiment of a neurostimulation application, the Injectrode® comprises a single distal region placed on or near the tissue target and a single proximal region placed right below the skin (subcutaneously). As described in the '007, '265 and '652 applications, these distal and proximal regions are bunched or bent into irregular shapes at a fixed depth, with fixed amounts of Injectrode® to be placed both near the tissue target and the subcutaneous region. The remainder of the Injectrode® length is within the central region which connects the distal and proximal regions. Placement depths and lengths must be controlled to ensure that consistent linear quantities of the flexible device are placed (in sufficient volume post-placement) in the distal and proximal regions. This capability is enabled by the calculation of interdependent markings on both the needle 33 and plunger 33A of the placement system. Stepwise advancement of the plunger and retraction of the needle to predetermined marks facilitate this controlled deployment of regions in a predictable manner.

In other embodiments, there are a plurality of distal regions and/or a plurality of proximal regions.

A variable depth placement is achieved by having a variable region centrally with fixed proximal and distal regions. The distance between the proximal and central points are fixed, and the volume of the irregular shapes which are the bunching anchor 8 and the connector pad 8A (respectively, the distal and proximal regions after placement) are fixed, while the volume of the central anchor 8B is variable in different use cases. In neurostimulation, for example a flexible medical device of a fixed length can be introduced to a variable depth target with a fixed location, such as the proximal region serving as a subcutaneous connector pad 8A for electrical coupling with a power source external to the body.

FIG. 1A is a schematic in an embodiment of a medical device with a single distal region and a single proximal region, showing the fully extended medical device 1, before insertion into the needle, and FIGS. 1B, 1C and 1D depict the relative size of three regions post-placement in the body, and a minimum to achieve the desired two fixed regions with one variable region. In FIG. 1B the central region is linear only, without an irregular shape or central anchor 8B. In FIGS. 1C and 1D there is a central anchor 8B, and its volume in 1C is smaller than in FIG. 1D, so the overall length of post-placement in FIG. 1C is shorter than in FIG. 1D. In other embodiments, one or more additional fixed regions can be provided for in certain use cases and can be accompanied in various embodiments by additional variable regions.

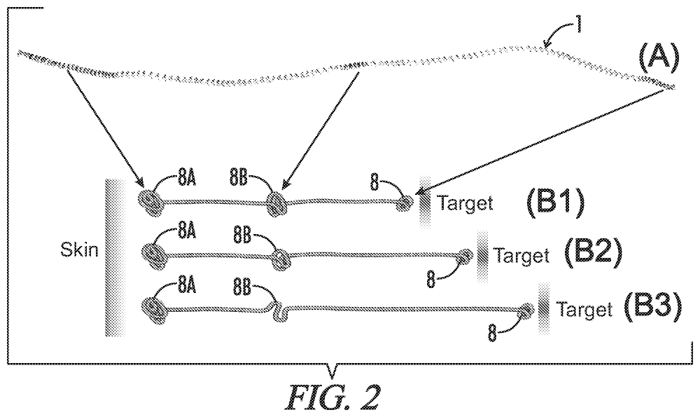

FIG. 2A is an image of an Injectrode®, in the same embodiment as in FIG. 1A-1D in its fully extended, pre-placement length. Note that it is substantially linear without any tines, hooks or other bent structures. Arrows point to the post-placement irregular shapes described in PCT/US2021/033007 (incorporated herein), which are also the distal anchor 8 and central anchor 8B and connector pad 8A shown schematically and in their position between the skin and the tissue target in FIGS. 2B1-2B3. All of the detail of the Injectrode® and its irregular shape, and the post-placement medical device is not drawn to scale, but the relative lengths as affected by volume of the central anchor are depicted. That is, relative length is a function of the volume of the central anchor 8B which declines from FIGS. 2B1 to 2B3. That is, for a given length pre-placement, the larger the central anchor 8B the shorter the length post-placement.

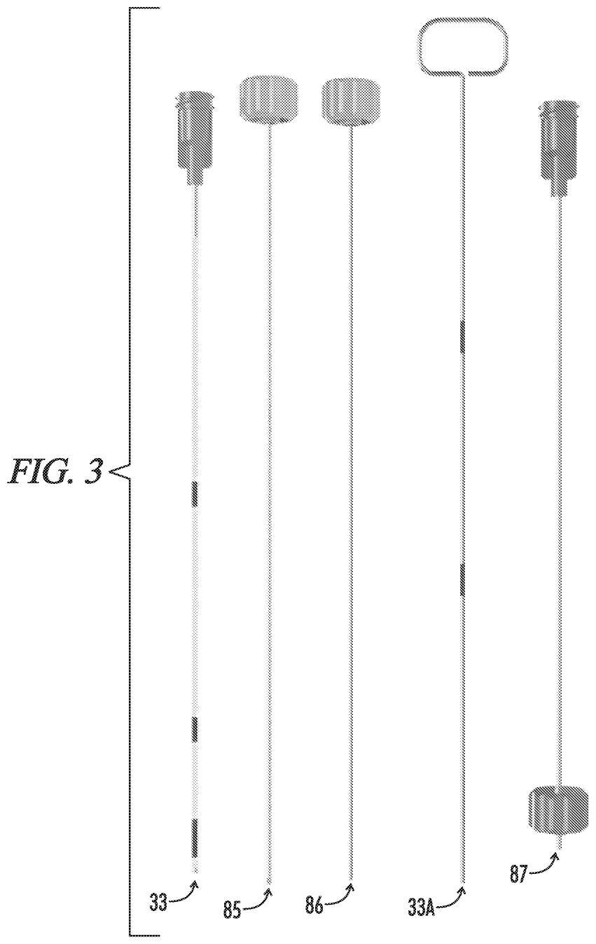

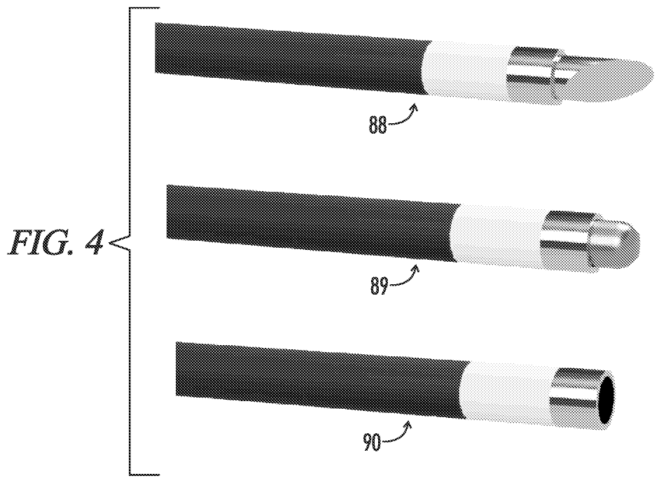

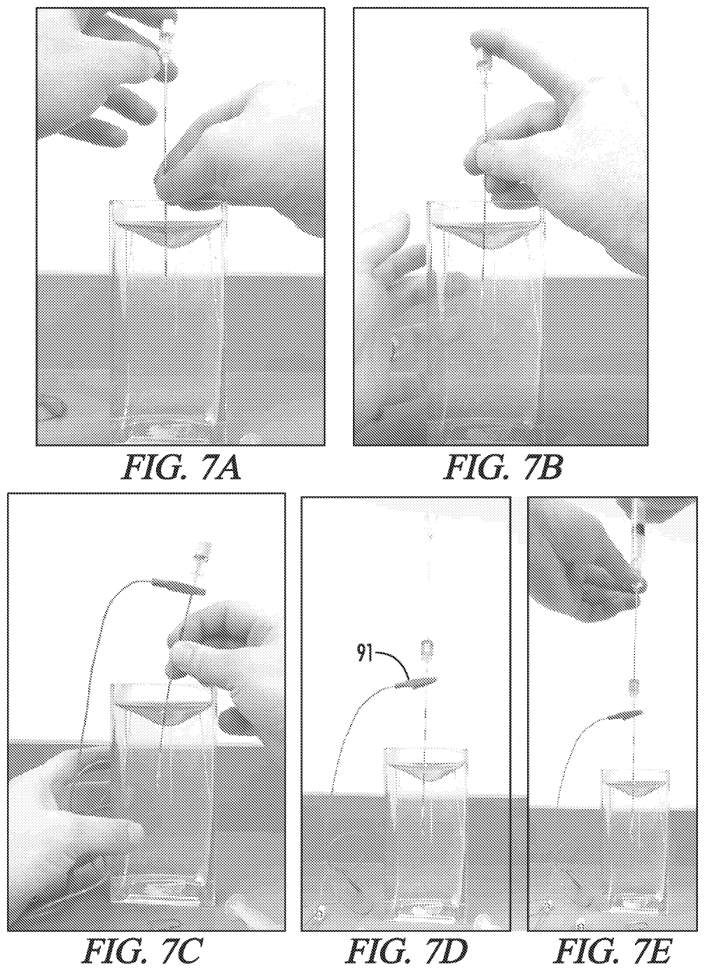

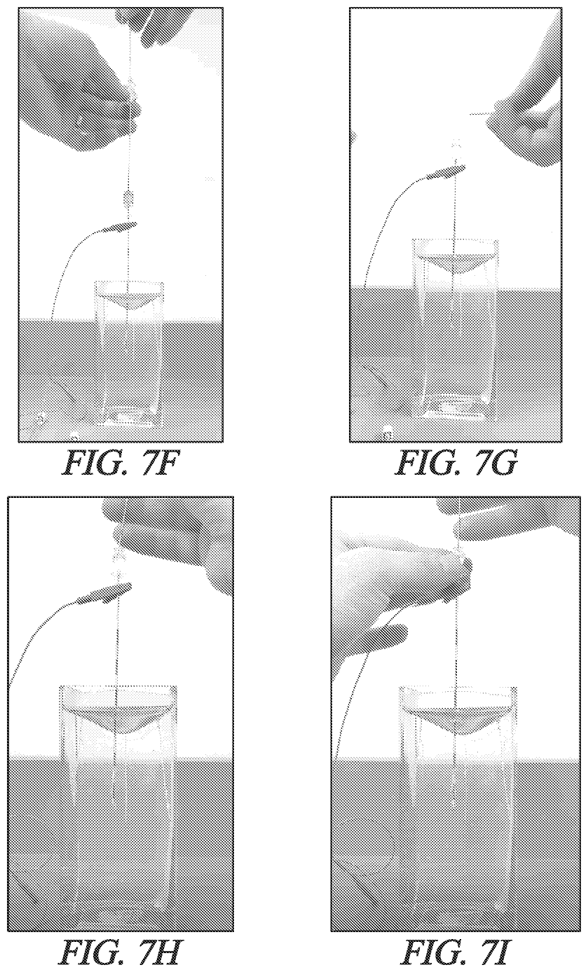

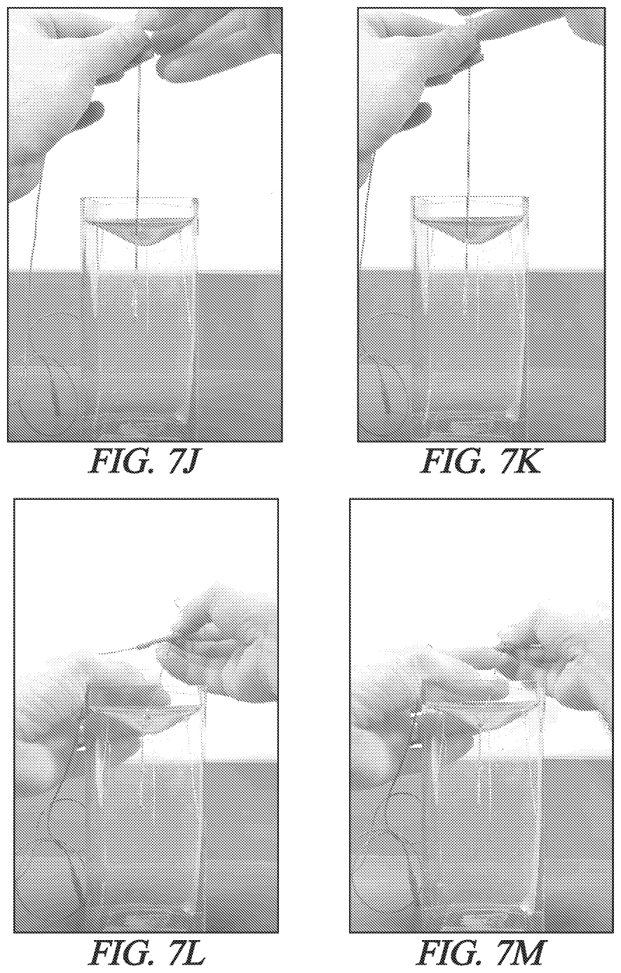

In one embodiment, the placement system comprises a needle 33 and plunger 33A with external markings. As shown in FIG. 3, in another embodiment the placement system comprises components including: (A) needle 33, (b) sharp trocar 85, (c) blunt trocar 86, (d) plunger 33A, and (e) transfer cannula 87. In another embodiment the system further comprises an electrical connection 91 (as shown in FIG. 7(C)-(H) to the needle to allow verification of contact with the tissue target. FIG. 4 contains three images depicting needle tip configurations: combined with a sharp trocar 88, combined with a blunt trocar 89, and with no trocar 90. In one embodiment the transfer cannula contains the flexible medical device to be placed. Both the needle and plunger have predetermined markings at distances either from the needle tip or the plunger base. In one embodiment the needle is partially coated but the needle base 33-1 and needle tip 33-2 (<1 mm) are left uncoated for electrical stimulation. In another embodiment the blunt trocar is partially coated, with the tip and an extended connection point being exposed for electrical stimulation or connection.

The placement system components mate together and attach via standard medical connectors, such as the luer lock system. Tubing for the needle and transfer cannula 87 has identical and concentrically aligned inner diameters. The sharp and blunt trocars 85, 86 fit smoothly within the needle and mate to the end with a connector to ensure that these components are secure during manipulation. The plunger 33A fits into the cannulas of both the needle 33 and transfer cannula. Prior to placement the transfer cannula containing the medical device is moved entirely into the needle.

Each flexible medical device to be placed has an overall length which totals the lengths for the at least one distal region (at the tissue interface), the at least one central region and the at least one proximal region (a subcutaneous connector pad 8 of current), and all of these lengths are predetermined for application/use and target. Markings (e.g., lines) on the needle and plunger allow the clinician to place the medical device in a precise position and configuration in the body.

During placement, the needle tip 33-2 should advance no farther below the skin than a maximum depth (max depth, or L1) which is the overall length of the medical device less substantially all the combined lengths of the distal and proximal regions. Max depth cannot be reached if a central anchor 8B is desired, i.e., the volume necessary for a central anchor 8B reduces the length of the implanted device. In the example of a placement without a central anchor 8B, first the clinician advances the needle tip to the max depth and next pushes the plunger to push the medical device from the transfer cannula so that it exits the needle tip.

The depths of the central region (central depth) and proximal region (proximal depth) are also predetermined by the surgical procedure and intended circumstances of use of the device. That is, in setting the central depth and the proximal depth, the maker of the system incorporates information on a range of shapes and dimensions for a tissue target of interest, as well as the tissue in the path to the tissue target from the skin, and other factors such as the amount of current needed

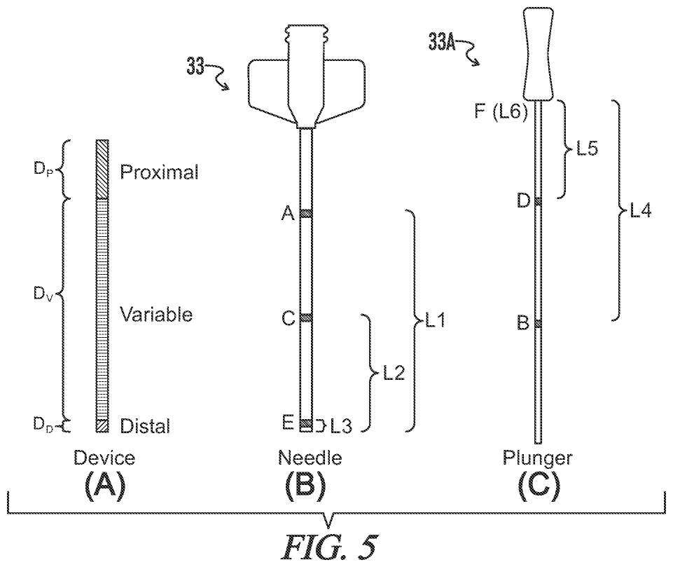

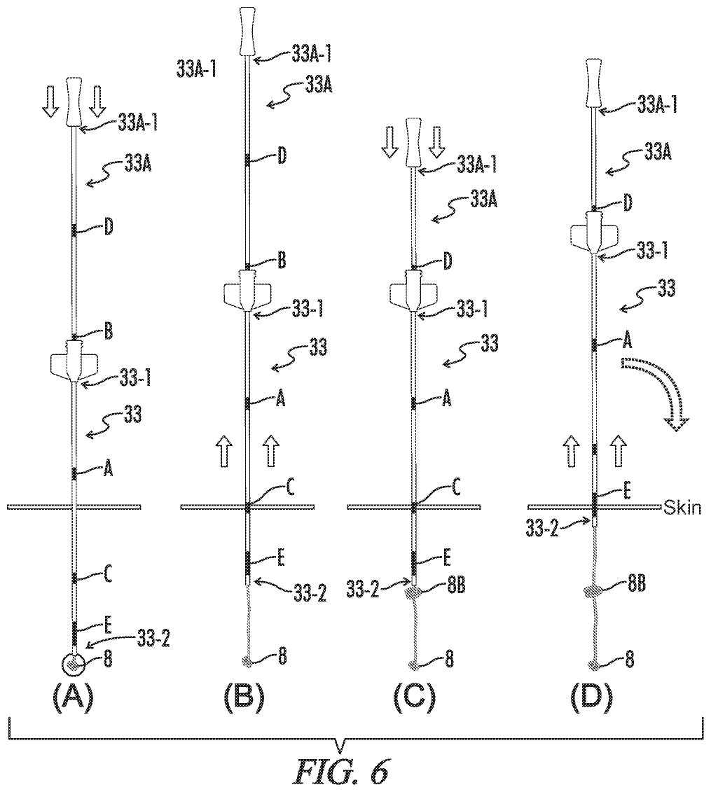

FIG. 5(A) represents a flexible medical device with three regions. In FIG. 5(B) the following markings on the needle representing a movement of the needle tip. Maximum depth L1 is a line from a maximum distal needle depth mark (point A) to the needle tip, meaning that the clinician inserts the needle to this mark. Central region depth L2 is a line from central region depth mark (point C) to the needle tip, indicating the beginning of ejection of the central region. Proximal region depth L3 is a line from proximal region depth (point E) to the needle tip. On FIG. 5C, there are the following markings on the plunger: from point B to the top of the plunger base is line L4, and from point D to the top of the plunger is line L5. Lines L4 and L5 represent forward movement of the plunger.

In Table 1, where
L1=Max Depth
L2=Central Depth
L3=Prox Depth

the following relations apply:

$$L 4 = L 1 + Proximal Length \\ L 5 = L 2 + Proximal Length$$

For various embodiments of the system for full implantation of a flexible medical device with a single distal region and a single proximal region, the dimensions in centimeters are as follows:

TABLE 1

| | | | | | | L4 | L5 |
|---|---|---|---|---|---|---|---|
| | | | L1 | L2 | L3 | (L1 + | (L2 + |
| Overall | Distal | Proximal | Max | Central | Proximal | Proximal | Proximal |
| Length | Length | Length | Depth | Depth | Depth | Length) | Length) |
| 10 | 1 | 2 | 7 | 2.5 | 0.5 | 9 | 4.5 |
| 7 | 1 | 2 | 4 | 1.5 | 0.5 | 6 | 3.5 |
| 13 | 1 | 2 | 10 | 4.5 | 0.5 | 12 | 6.5 |

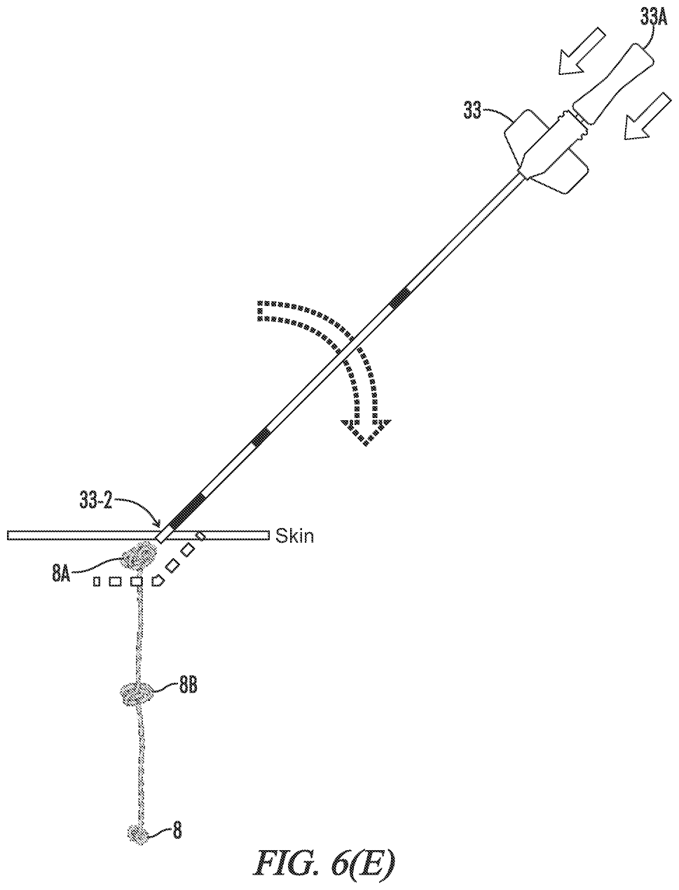

The relative positions of the needle and plunger as placement proceeds are depicted in FIG. 6(A)-(E). In 6(A) the needle tip has already advanced to the maximum distal needle depth mark at point A and the medical device has been ejected creating the distal anchor 8 through pushing the plunger 33A to point B. In 6(B), the needle tip has been withdrawn further to a central region depth mark at point C creating a substantially linear portion of the device. In 6(C) the plunger has advanced to a central region advance mark at point D, thereby injecting the central anchor region 8B. In 6(D), the needle has been withdrawn to the proximal region depth mark at point E. In 6(E) the plunger has been advanced to deploy the proximal region as a connector pad 8A in the subcutaneous space below the skin, and the lateral movement of the needle and plunger can create a dimension more parallel to the skin which is wider and allows more area for electrical coupling with an external power source positioned on the skin.

Needle and plunger markings for the placement of a three region device for which the Distal region 1A is the “Stimulator” 8, the Central region 1B is the “Lead” and the Proximal region 1C is the “Connector pad” 8A. The distances as labeled correspond to the distances from either the needle tip 33-2 or plunger base 33A-1 as indicated for A=L1, B=L4, C=L2, D=L5, and E=L3. Generally point F (L6) is left at a distance of zero relative to the plunger base 33A-1 to enable complete deployment of the device, whereas a non-zero distance would result in partial deployment of the proximal region 1C if desired. Point F is a proximal advance mark, and with multiple proximal anchors and regions there are multiple proximal advance marks as in FIG. 11(C).

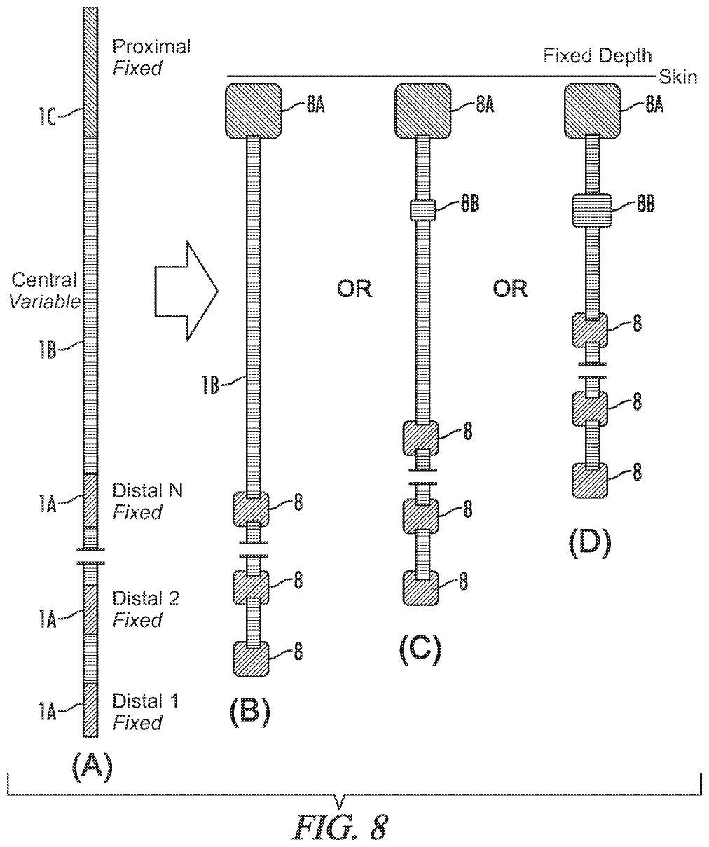

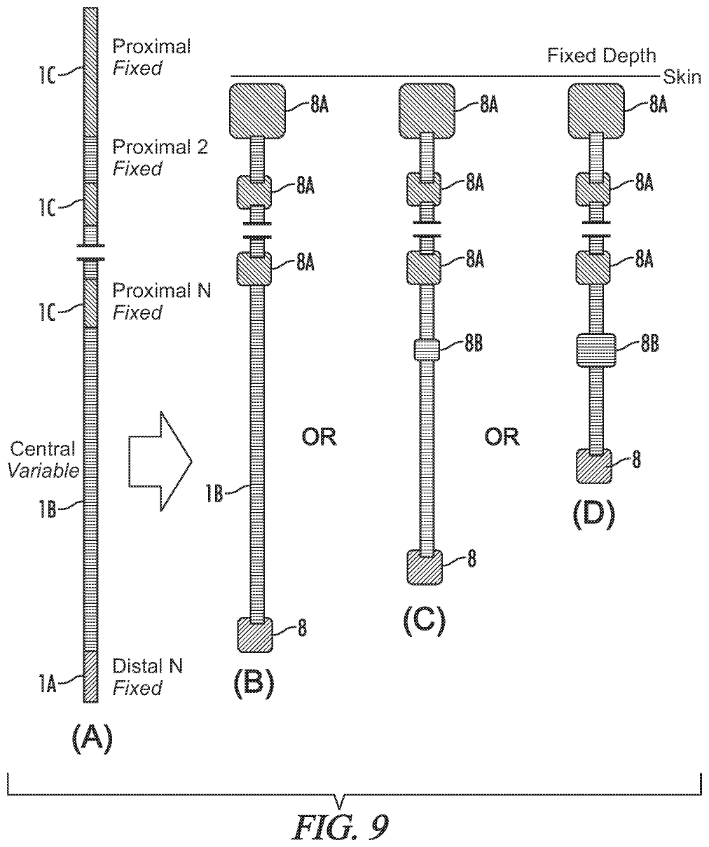

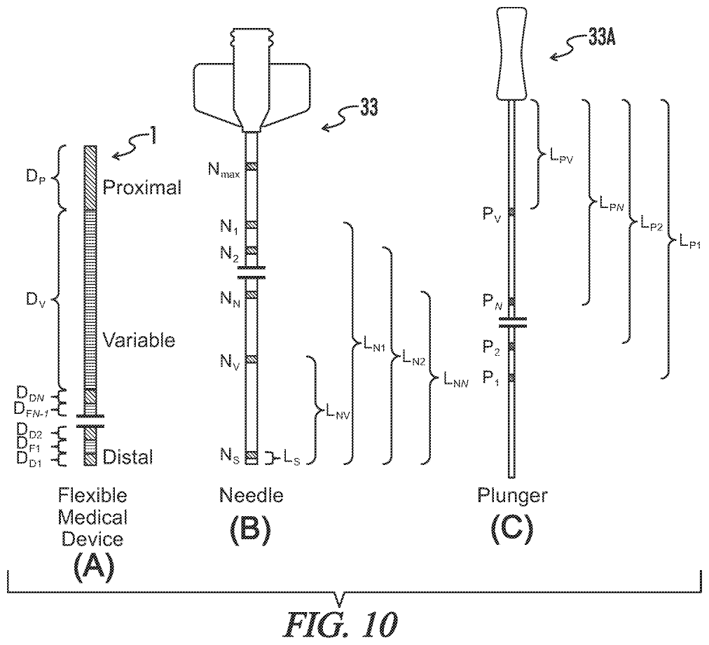

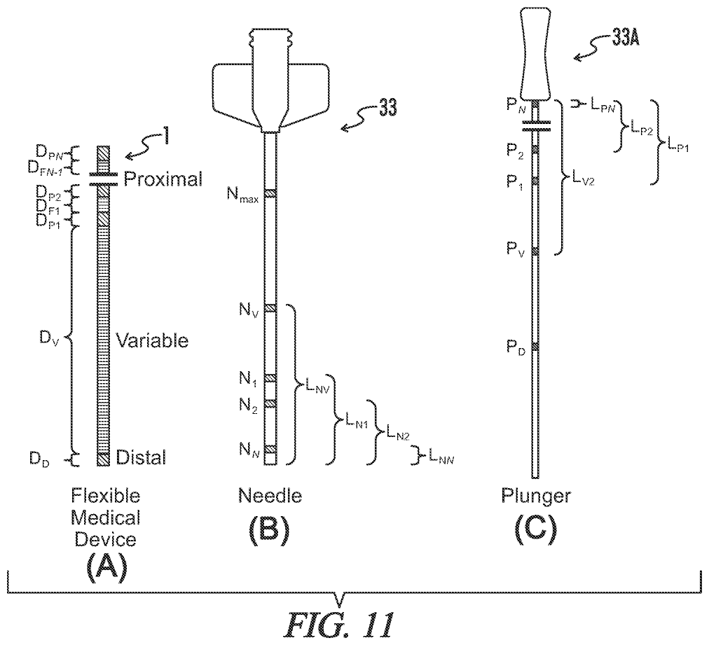

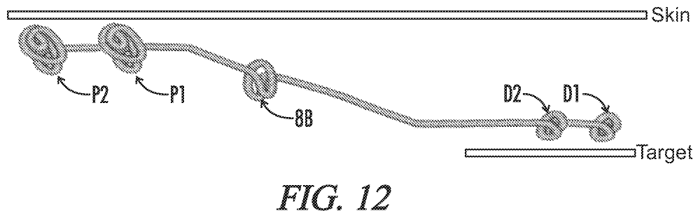

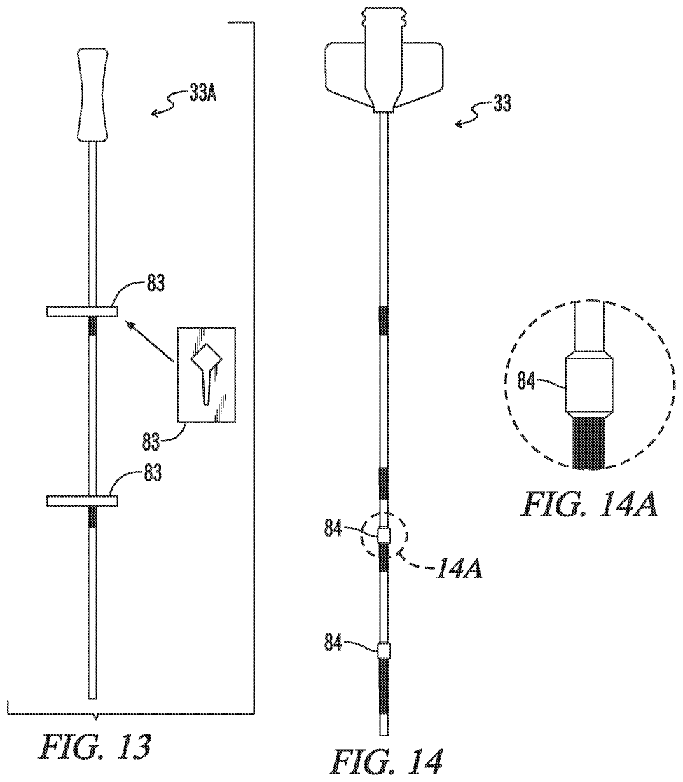

Usage of the placement system with the following methods enables placement of the controlled and distinct bunched/bundled regions of the flexible medical device. The use of liquid renders the compressible device less compressible due to the non-compressibility of the liquid surrounding the injectable device within the enclosed cannula of the needle. The placement methods as described herein achieve regional fixation for a fully implanted flexible medical device, as shown in a benchtop setting in FIG. 7(A)-(M):
1. Using the sharp trocar within the needle, puncture the skin and advance through the muscle planes or other difficult to dissect tissues
2. Replace the sharp trocar with the blunt trocar within the needle and further advance the needle tip towards the tissue target, at a maximum distal needle depth mark (point A)
3. Optionally for neurostimulation applications, a stimulating alligator clip may be attached to the base of the uncoated region of the needle or blunt trocar to stimulate the tissue target and confirm location
4. Remove the blunt trocar and replace with the transfer cannula containing the medical device to be injected
5. Optionally, the clinician may prime the device with an excipient injection via a syringe at the back end of the transfer cannula.
6. Using the plunger, transfer the medical device from the transfer cannula into the needle.
7. Remove the transfer cannula and plunger and confirm that the medical device has been successfully transferred. At this time additional stimulation via the attached alligator clip may be undertaken to re-confirm location and no shifting during transfer.
8. Insert the plunger into the base of the needle.
9. Advance the plunger to the distal region advance mark (point B) to eject the fixed distal region(s) of the medical device. In embodiments with more than one distal region (as in FIG. 8) this is repeated as shown in FIG. 10.
10. Withdraw the needle to central region depth mark (point C) for the central region anchoring site.
11. Advance the plunger to central region advance mark (point D), thereby injecting the Central anchoring region of the medical device. In embodiments with more than one central region/anchor, this is repeated.
12. Withdraw the needle to the proximal region depth mark (point E).
13. Advance the plunger fully to deploy the fixed proximal region(s) of the medical device at the proximal advance mark (point F). In embodiments with more than one proximal region (as in FIG. 9) this is repeated as shown in FIG. 11.

Another embodiment of the invention herein enables placement of a flexible medical device partially in the body and to leave an external tail above the skin, or an external loop whose end may be implanted in the skin to provide more stability for the external portion through tissue ingrowth around the end. In one embodiment, the plunger is shorter than the length of the needle by the length desired for the external tail. This externalization length difference is subtracted from the distal region advance mark (point B) and central region advance mark (point B). Here the needle tip is removed from the skin after the plunger is fully inserted in the last step, so the proximal end of the flexible medical device, still in the needle, can then be ejected so that it is outside the skin.

The placement methods as described herein achieve regional fixation for a partially implanted flexible medical device, that is, having an external tail or loop. In one embodiment the steps comprise:
1. Using the sharp trocar within the needle, puncture the skin and advance through the muscle planes or other difficult to dissect tissues
2. Replace the sharp trocar with the blunt trocar within the needle and further advance the needle tip towards the tissue target, at the at least one maximum distal needle depth mark (point A)
3. For neurostimulation applications, a stimulating alligator clip may be attached to the base of the uncoated region of the needle or blunt trocar to stimulate the tissue target and confirm location
4. Remove the blunt trocar and replace with the transfer cannula containing the medical device to be injected
5. Prime the device with an excipient injection via a syringe at the back end of the transfer cannula
6. Using the plunger, transfer the medical device from the transfer cannula into the needle.
7. Remove the transfer cannula and plunger and confirm that the medical device has been successfully transferred. At this time additional stimulation via the attached alligator clip may be done to re-confirm location and no shifting during transfer.
8. Insert the plunger into the base of the needle.
9. Advance the plunger to the at least one distal region advance mark (point B) to eject the fixed Distal region of the medical device.
10. Withdraw the needle to central region depth mark (point C) for the central region anchoring site.
11. Advance the plunger to the at least one central region advance mark (point D), thereby injecting the central anchor of the medical device.
12. Optionally withdraw the needle to the proximal depth mark (point E) and advance the plunger to eject a proximal region of the medical device in a subcutaneous region at the at least one proximal advance mark (point F).
13. Withdraw the needle from the skin and then advance the plunger fully to eject an external tail of the medical device outside the skin.
14. Optionally implant in the skin an external end of the medical device to create a loop implanted on both sides.

In other embodiments the flexible medical device may have two or more distal regions (FIG. 8(A)-(D) and/or two or more proximal regions (FIG. 9(A)-(D), and so the placement system and methods are adjusted accordingly. The lengths of the distal or proximal regions may be the same or different, but they are predetermined and therefore fixed in length, and have additional fixed lengths of the medical device between each distal or proximal region. As illustrated, a break is shown in the device for both the linear and post-placement examples (FIG. 8(A)-(D), (FIG. 9(A)-(D)) by two horizontal lines between the second distal or proximal region (Distal 2 Fixed, Proximal 2 Fixed) and the Nth distal or proximal region (Distal N Fixed, Proximal N Fixed). The flexible medical device may comprise both two or more distal and proximal regions by combining the distal and proximal regions above and below the central variable region.

An embodiment of the invention herein enables the placement of a flexible medical device in FIG. 8(A)-(D) with two or more distal regions. FIG. 10(A) shows the medical device (also incorporating an interdistal region), and FIGS. (B) and (C) are distances and markings on the needle and plunger to enable placement of a device with N distal regions. The lengths of each distal region is labeled D_{D1}, D_{D2} through D_{DN}. Between each fixed distal region is another fixed region that dictates the inter distal region spacing upon placement, and is noted as D_{F1}, D_{F2} through D_{FN-1}. N_{MAX} is the maximum distal needle depth marking on the needle. This marking is followed by markings N1, N2 NN, denoting additional maximum distal needle depths at which each concessive distal region is placed and has a total distance from the needle tip of L_{N1}, L_{N2} through L_{NN} respectively. Retracting the needle up to each concessive needle marking starting from N1 therefore denotes the maximum depth at which each distal region is placed. N_{V} corresponds to the central depth at a distance of L_{NV} from the needle tip. The proximal depth marking, NS, has a distance of L_{S} from the needle tip. Similarly, the plunger has multiple distal advance markings P₁, P₂ through P_{N} to advance the medical device for each corresponding distal region, with distances from the base of the plunger denoted as L_{P1}, L_{P2} through L_{PN}. The variable region placement marking on the plunger, P_{V}, is at a length of L_{PV} from the plunger base. The relationships between the length and distance variables can be written as follows to achieve the placement of multiple distal regions at variable overall depth (FIG. 11):

$${N}_{MAX} = {D}_{V} + {\sum }_{x = 1}^{N} {D}_{F x}$$

For each successive y-th marking on the needle up to the N-th marking:

$${L}_{Ny} = {L}_{NV} + {\sum }_{x = y}^{N} {D}_{Fx}$$

For each successive y-th marking on the plunger up to the N-th marking:

$${L}_{Py} = {D}_{P} + {D}_{V} - {D}_{Dy} + {\sum }_{x = y}^{N} ( {D}_{Dx} + {D}_{Fx} )$$

An embodiment of the invention which enables the placement of a flexible medical device in FIG. 9(A) with two or more proximal regions 1C. FIG. 11(A) shows the medical device (also incorporating an interproximal region), and FIGS. 11(B) and (C) are distances and markings on the needle and plunger to enable placement of a device with N distal regions. Calculations for the proximal region markings follow a similar mathematical schema for the markings for multiple distal regions (FIG. 10A-10C). For multiple proximal regions, the markings are instead between the central depth marking (N_{V}) and the needle tip and between P_{V} and the plunger base 33A-1. The lengths of each proximal region are noted as D_{P1}, D_{P2} through D_{PN}. Between each fixed proximal region is another fixed region that dictates the inter proximal region spacing upon placement, and is noted as D_{F1}, D_{F2} through D_{FN-1}. N_{MAX} is the maximum depth marking on the needle. This marking is followed by the variable depth marking N_{V} at a distance of L_{VN} from the needle tip corresponding to the central variable region depth. Proximal depth markings N1, N2 through NN, denote the minimum depths at which each concessive proximal region is placed and has a total distance from the needle tip of L_{N1}, L_{N2} through L_{NN} respectively. Retracting the needle up to each concessive needle marking starting from N1 therefore denotes the minimum depth at which each proximal region is placed. LMIN represents the minimum depth at which the last proximal region is to be placed, and is determined based on clinical application and use of the placed device.

Similarly, the plunger has proximal advance markings P₁, P₂ through P_{N} to advance the medical device for each corresponding proximal region, with distances from the base of the plunger denoted as L_{P1}, L_{P2} through L_{PN}. The variable region placement marking on the plunger, P_{V}, is at a length of L_{PV} from the plunger base. The distal region plunger advance marking P_{D}, is at a distance of L_{PD} from the base of the plunger. The relationships between the length and distance variables can be written as follows to achieve the placement of multiple proximal regions (FIG. 6):

$${N}_{MAX} = {D}_{V} + {\sum }_{x = 1}^{N} {D}_{Fx}$$

For each successive y-th marking on the needle up to the N-th marking:

$${L}_{Ny} = {L}_{MIN} + {\sum }_{x = y}^{N} {D}_{Fx}$$

For each successive y-th marking on the plunger up to the N-th marking:

$${L}_{Py} = - {D}_{Py} + {\sum }_{x = y}^{N} ( {D}_{Px} + {D}_{Fx} )$$

Therefore, at a value of y=N, Ly will be zero since DEN is undefined and therefore also zero. Should an externalized “tail” be desired, the overall length of the plunger will be reduced to match the desired length of “tail”, which is not to exceed the length of D_{PN} on the device.

Although not depicted, there may be multiple central anchors placed with multiple central depth marks on the needle and multiple central advance marks on the plunger, in a manner analogous to the multiple distal and proximal marks.

FIG. 12 is a schematic showing how a flexible medical device with two distal regions are placed near a tissue target with two distal anchors, two proximal regions are positioned subcutaneously with two proximal anchors (connector pads), the distal and proximal regions connected by a central region with a central anchor.

For tactile or additional non-visual cues to alert a user to the successive markings, the needle and plunger are modified. The plunger 33A receives clips that secure at the markers of the plunger by a friction fit (FIG. 13). Advancement of the plunger is blocked by successive securing clips 83, which need to be removed or forced aside to further advance the plunger into the needle. The needle has projections 84, either swaged, welded, coated, brazed or the like, to expand the diameter at or prior to the markings on the needle (FIG. 14). Retraction or advancement of the needle past these projections in the diameter provide the user with tactile feedback as additional force is required to advance the needle through the skin at the projections 84.

1. A system for placement of a flexible medical device in a body comprising: a needle with needle position markings, a plunger bearing one or more plunger advance marks, and a transfer cannula containing the flexible medical device, configured to guide a clinician to place said flexible medical device in a precise location and configuration in said body.

2. The system as in claim 1 wherein said one or more needle position markings include at least one maximum distal needle depth mark, at least one central region depth mark, and at least one proximal region depth mark.

3. The system as in claim 1 wherein said one or more needle position markings include a at least one maximum distal needle depth mark and at least one central region depth mark.

4. The system as in claim 1 wherein said one or more plunger advance marks include at least one distal region advance mark, at least one central region advance mark and at least one proximal advance mark.

5. The system as in claim 1 wherein said needle and said plunger contain tactile features to indicate at least one of the one or more needle position markings and/or plunger advance marks.

6. The system as in claim 1 configured for placing multiple distal anchors located near a tissue target.

7. The system as in claim 1 further configured for placing multiple connector pads to be placed in a subcutaneous region of said body.

8. (canceled)

9. (canceled)

10. (canceled)

11. The system of claim 7, wherein the one or more connector pads are fixed and configured for electrical coupling with a power source external to the body.

12. The system of claim 1, further comprising:
a substantially linear portion of the flexible medical device to be oriented inside or outside of a tissue target or exterior to a patient's skin.

13. A method for full placement of a flexible medical device below a patient's skin including the steps of: providing a system comprising: a needle with one or more needle position markings and a needle tip, the needle is configured to eject the flexible medical device; and a plunger bearing one or more plunger advance marks; advancing the needle tip of the needle below the patient's skin towards a tissue target to a maximum distal needle depth mark of the one or more needle position markings on the needle; partially advancing the plunger within the needle to a distal region advance mark of the one or more plunger advance marks on the plunger and ejecting a fixed distal region of the flexible medical device; partially withdrawing from the patient's skin, the needle tip of the needle, to a central region depth mark of the one or more needle position markings on the needle; partially advancing the plunger to a central region advance mark of the one or more plunger advance marks on the plunger, and ejecting a central anchor region of the flexible medical device; partially withdrawing from the patient's skin, the needle tip of the needle, to a proximal region depth mark of the one or more needle position markings on the needle; advancing the plunger fully to eject a fixed proximal region of the flexible medical device; and removing the needle tip from the patient's skin.

14. The method of claim 13, wherein the fixed distal region of the flexible medical device is a distal anchor.

15. The method of claim 14, wherein the distal anchor has an irregular shape.

16. The method of claim 13, further comprising:
a substantially linear portion of the flexible medical device is located between the fixed distal region and the central anchor region.

17. The method of claim 13, wherein the central anchor region is an irregular shape.

18. The method of claim 13, wherein the fixed proximal region of the flexible medical device is a subcutaneous connector pad configured for electrical coupling with a power source external to the patient's skin.

19. The method of claim 13, wherein the advancing the plunger fully to eject the fixed proximal region of the flexible medical device further comprising:
orienting the needle and the plunger in a lateral position in relation to the patient's skin.

20. The method of claim 13, further comprising:
orienting a substantially linear portion of the flexible medical device inside or outside of the tissue target or exterior to the patient's skin.

21. A method for partial placement of a flexible medical device below a patient's skin including the steps of: providing a system comprising:
a needle with one or more needle position markings and a needle tip, the needle is configured to eject the flexible medical device; and a plunger bearing one or more plunger advance marks; advancing the needle tip of the needle below the patient's skin towards a tissue target to a maximum distal needle depth mark of the one or more needle position markings on the needle; partially advancing the plunger within the needle to a distal region advance mark of the one or more plunger advance marks on the plunger and ejecting a fixed distal region of the flexible medical device; partially withdrawing from the patient's skin, the needle tip of the needle, to a central region depth mark of the one or more needle position markings on the needle; partially advancing the plunger to a central region advance mark of the one or more plunger advance marks on the plunger, and ejecting a central anchor region of the flexible medical device; withdrawing the needle tip of the needle from the patient's skin; and advancing the plunger fully to eject an external region of the flexible medical device located external to the patient's skin.

22. The method of claim 21, wherein the fixed distal region of the flexible medical device is a distal anchor.

23. The method of claim 21, wherein the distal anchor has an irregular shape.
a. advancing the plunger to a central region advance mark on the plunger, and ejecting a central anchoring region of the flexible medical device,
b. withdrawing the needle tip from the skin,
c. advancing the plunger fully to eject an external region of the flexible medical device outside the skin.
